(19) United States
(12) Patent Application Publication
(10) Pub. No.: US 2026/0127680 A1
(43) Pub. Date: May 7, 2026

(54) ELECTRONIC MEDICAL RECORDS SYSTEMS TRANSITIONS TO SUPPORT PENDING INSURANCE CLAIMS

(71) Applicant: Ocean Friends, Inc., Irving, TX (US)

(72) Inventors: Anusha Tiwari, Chennai, TN (US); Manish Shukla, North Bend, WA (US)

(73) Assignee: Ocean Friends, Inc., Irving, TX (US)

(21) Appl. No.: 19/380,942

(22) Filed: Nov. 5, 2025

Related U.S. Application Data
(60) Provisional application No. 63/716,721, filed on Nov. 5, 2024.

(51) Int. Cl. G06Q 40/08; G16H 10/60

(52) U.S. Cl. CPC G06Q 40/084; G16H 10/60

(57) ABSTRACT

An automatic bulk insurance claims re-submission of pending insurance claims facilitates transitioning from one technical electronic medical record (EMR) system to another EMR system. The method involves extracting past insurance claims that remain pending due to non-submission or rejection by one or more payers for various reasons, classifying these as pending insurance claims. It further includes analyzing Electronic Remittance Advice (ERA) reconciliation data provided by the payer to identify discrepancies between expected and actual payments. The payment status and reasons behind non-payment for each pending claim are determined. Necessary modifications are then implemented to ensure that the claims meet approval criteria. Finally, the method automatically resubmits all modified pending claims in a single bulk upload, ensuring that all corrected claims are accurately reflected in the submission, and ensuring that the efficiency and accuracy of the insurance claim re-submission process are maintained, leading to improved reimbursement outcomes for healthcare providers.

### CROSS-REFERENCE TO RELATED APPLICATION(S)

This application claims the benefit under 35 U.S.C. § 119(e) and 37 C.F.R. § 1.78 of U.S. Provisional Application No. 63/716,721, which is incorporated by reference in its entirety.

### FIELD OF THE INVENTION

The present invention generally relates to the field of electronics, and more specifically to the automatic re-submission of electronic medical records systems transitions to support pending insurance claims.

### BACKGROUND

Health insurance claim forms are standard documents used by healthcare providers. They contain the necessary patient and treatment information and help request payment from health insurance companies. Health insurance claim forms are vital for billing and claims processing systems. The claim forms provide clear communication regarding medical services availed, the scope of the services, and the cost involved. Healthcare providers use them to get reimbursed accurately and promptly. For insurance companies, the data helps in fraud detection, claim processing, and reimbursements.

Billing details are crucial for healthcare providers and insurance companies. The data ensures that healthcare providers are reimbursed correctly for their services, and insurance companies can use it to verify coverage and expedite reimbursement. Accurate financial information helps prevent billing errors and claim denials and facilitates smoother cash flow between all parties involved.

Conventionally, the process of handling health insurance claims has several limitations. These methods do not include the analysis of Electronic Remittance Advice (ERA) data, which is essential for identifying discrepancies between expected and actual payments. As a result, claims that remain unpaid or are incorrectly processed often go unnoticed, leaving healthcare providers with unresolved pending claims. Moreover, when claims are rejected or denied, the process of identifying the reasons for rejection or non-payment typically involves manual checking. This manual process is time-consuming and prone to errors, leading to delays in resolving issues and potentially resulting in lost revenue for healthcare providers.

Additionally, leftover claims that are neither submitted nor addressed after an initial submission failure are often left as they are, without further action. This inaction can lead to significant financial losses and inefficiencies in the billing process. For healthcare providers, accurate billing details are crucial not only for receiving proper compensation for services rendered but also for maintaining smooth cash flow. For insurance companies, this data aids in verifying coverage, detecting potential fraud, and ensuring prompt reimbursement. Accurate financial information helps prevent billing errors and claim denials, which are common issues in the healthcare billing ecosystem.

### SUMMARY

A bulk insurance claims re-submission system and process is disclosed herein which deals with the re-submission of pending insurance claims, that may have been denied or otherwise rejected, to the payer by uploading all the pending insurance claims in bulk.

In an embodiment of the present disclosure, an automatic bulk insurance claims re-submission method is disclosed. The automatic bulk insurance claims re-submission method for the pending insurance claims is disclosed. The automatic bulk insurance claims re-submission method involves extracting past insurance claims that remain pending due to non-submission, denial or rejection by one or more payers for various reasons, classifying these as pending insurance claims. Further, the Electronic Remittance Advice (ERA) reconciliation data provided by the payer is analyzed to identify discrepancies between expected and actual payments. The payment status and reasons behind non-payment for each pending claim are determined. A set of necessary modifications are then implemented to ensure that the claims meet approval criteria. Finally, the automatic bulk insurance claims re-submission method automatically resubmits the modified pending claims in a single bulk upload, ensuring that all corrected claims are accurately reflected in the submission, and ensuring that the efficiency and accuracy of the insurance claim re-submission process are maintained, leading to improved reimbursement outcomes for healthcare providers.

In addition, an automatic bulk insurance claims re-submission system is disclosed. The automatic bulk insurance claims re-submission system for automatic bulk re-submission of pending insurance claims includes a computer system with one or more processors and databases, operatively coupled to the processors, to perform specific operations. “Pending” claims includes non-submitted claims and submitted but rejected claims. The automatic bulk insurance claims re-submission system extracts past insurance claims that are pending, either due to non-submission, denial or rejection by one or more payers for various reasons, classifying these as pending insurance claims using a data collector. The Electronic Remittance Advice (ERA) reconciliation data provided by the payer is analyzed through an analyzer, identifying discrepancies between expected and actual payments. The automatic bulk insurance claims re-submission system also determines the payment status of each pending claim and the reasons for non-payment using a payment checker. A set of necessary modifications are implemented using an insurance claim modifier to ensure the claims meet the criteria for approval upon re-submission. Finally, the automatic bulk insurance claims re-submission system automatically re-submits the modified pending claims to the payers in a single bulk upload using a bulk uploader, ensuring accurate and complete submission of corrected claims.

Furthermore, the errors and discrepancies due to which the insurance claim is pending and requires modifications are automatically detected and flagged based on the analyzed ERA reconciliation data.

Further, the pending insurance claims are visible to the user or healthcare provider on a user interface integrated into the online billing platform.

### BRIEF DESCRIPTION OF THE DRAWINGS

The systems and methods described herein may be better understood, and their numerous objects, features, and advantages are made apparent to those skilled in the art by referencing exemplary embodiments depicted in the accompanying figures. The use of the same reference number throughout the several figures designates a like or similar element.

FIG. 1 depicts an exemplary automatic bulk insurance claims re-submission system.

FIG. 2 depicts an exemplary automatic bulk insurance claims re-submission process.

FIG. 3 depicts an exemplary user interface disclosing the user profile categorized based on various parameters.

FIG. 4 depicts an exemplary user interface disclosing the list of pending insurance claims.

FIG. 5 depicts an exemplary user interface that discloses the reason for the rejection or denial of the pending insurance claim.

FIG. 6 depicts an exemplary user interface that discloses the modified insurance claim form by the user.

FIGS. 7 and 8 depict exemplary user interfaces that refresh the pending insurance claim form after modification and submit it to the payer respectively.

FIG. 9 depicts an exemplary network environment in which the system of FIG. 1 and the process of FIG. 2 may be practiced.

FIG. 10 depicts an exemplary computer system.

### DETAILED DESCRIPTION

An automatic bulk insurance claims re-submission system is disclosed. Barriers exists that often prevents licensees of electronic medical record (EMR) systems (also sometimes referred to as electronic health record (EHR) systems) from transitioning from one EMR system provider to another. EMR systems record provision of medical services and submit the corresponding charges to insurance companies for reimbursement. At least one of the barriers relates to a lag in time between provision of medical services provided and submission of an insurance reimbursement request or corresponding payment receipt. If a licensee, such as a medical clinic owner, hospital, or other medical service provider transitions from one EMR system provider to another, records of the medical services provided may be transferred but pending claims, either due to non-submission or payment rejection, may not be tracked and/or processed. Accordingly, medical service providers are often reluctant to transition from one EMR system to another. The systems discussed herein provide a technical solution that allows medical service providers to transition from one EMR system to another. Accordingly, in at least one embodiment, an intermediary computer system uploads and processes the insurance medical records and insurance claims from a first electronic medical records system to the computer system to facilitate transitioning from the first electronic medical records system to a second electronic medical records system. In at least one embodiment, the bulk insurance claims re-submission system is integrated with a second EMR system.

The automatic bulk insurance claims re-submission system for automatic bulk re-submission of pending insurance claims includes a computer system with one or more processors and memory, operatively coupled to the processors, to perform specific operations. The automatic bulk insurance claims re-submission system further comprises an online billing platform and a bulk insurance claim re-submission module operatively coupled to each other.

The bulk insurance claim re-submission module includes a data collector that extracts past insurance claims that are pending, either due to non-submission, denial or rejection by one or more payers for various reasons. The Electronic Remittance Advice (ERA) reconciliation data provided by the payer is analyzed through an analyzer, identifying discrepancies between expected and actual payments. Further, a payment checker integrated within the bulk insurance claim re-submission module determines the payment status of each pending claim and the reasons for non-payment. A set of necessary modifications are implemented using an insurance claim modifier to ensure the claims meet the criteria for approval upon re-submission.

Finally, a bulk uploader operatively coupled to the online billing platform and the bulk insurance claim re-submission module automatically re-submits the modified pending claims to the payers in a single bulk upload, ensuring accurate and complete submission of corrected claims.

The automatic bulk insurance claims re-submission system offers significant advantages in the healthcare billing and insurance claim process by integrating automated systems for identifying, reconciling, and resubmitting pending claims. By utilizing ERA data analysis and machine learning algorithms, the bulk insurance claims re-submission system can detect and flag discrepancies and errors that might lead to claim rejections and denials, reducing the need for manual intervention and minimizing human error. The automated extraction and modification of claim details ensure that claims are accurate and complete, leading to higher approval rates and faster reimbursements. Additionally, the notification module keeps healthcare providers and users informed about claim statuses and required actions, improving communication and efficiency.

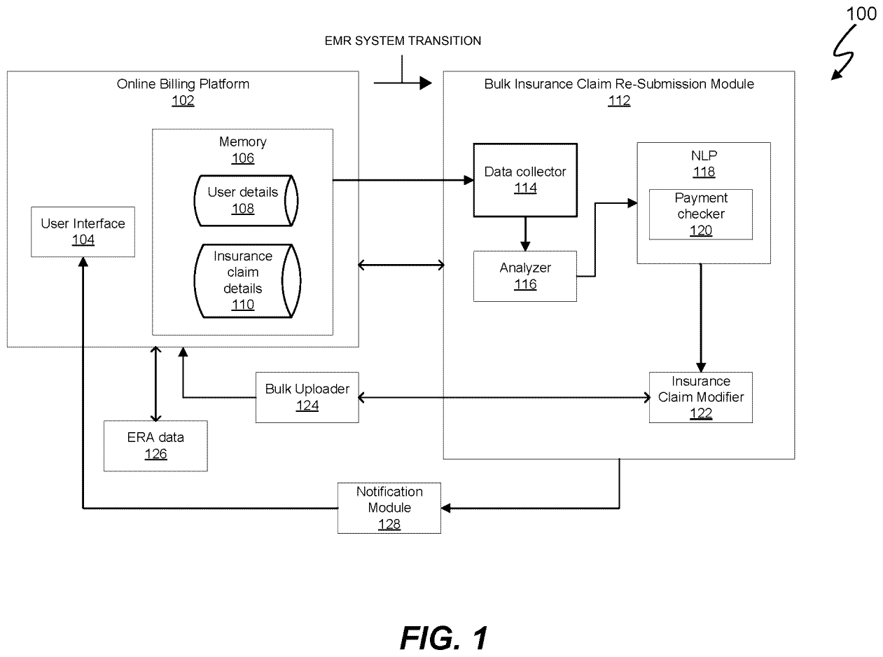

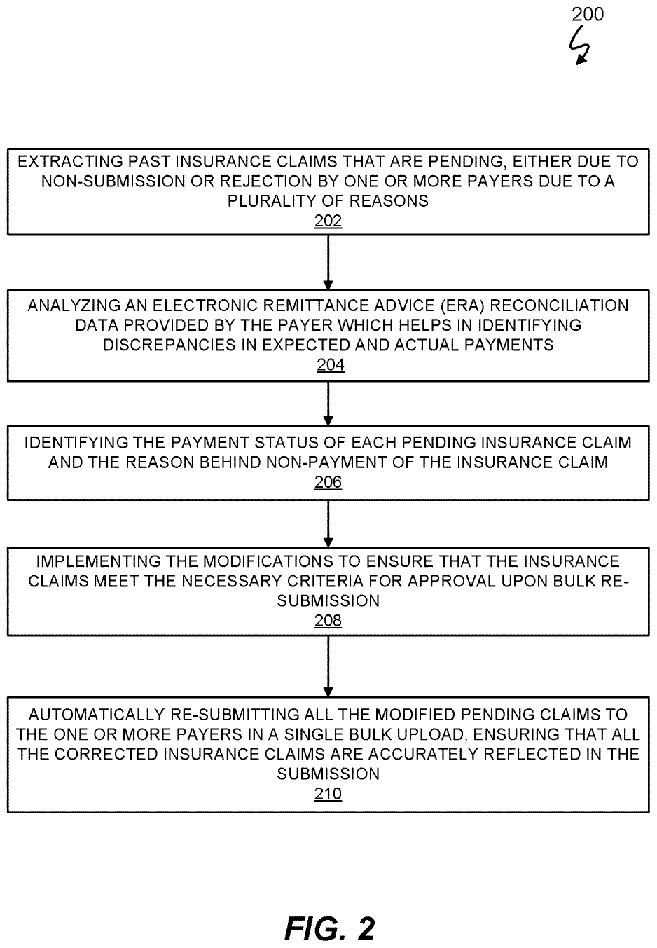

FIG. 1 depicts an exemplary automatic bulk insurance claims re-submission system 100. FIG. 2 depicts an exemplary automatic bulk insurance claims re-submission process 200 utilized by the automatic bulk insurance claims re-submission system 100. Referring to FIGS. 1 and 2, to facilitate transitioning from the first electronic medical records system to a second electronic medical records system, in operation 202, a data collector 114 extracts past insurance claims that are pending, either due to non-submission or rejection by one or more payers due to a plurality of reasons. The non-submitted, rejected, or denied insurance claims are classified as pending insurance claims. In at least one embodiment, prior to operation 202, the data collector 114 uploads at least pending insurance claims from a first electronic medical records system to a computer system to facilitate transitioning from the first electronic medical records system to a second electronic medical records system.

The data collector 114 is integrated within a bulk insurance claim re-submission module 112. The bulk insurance claim re-submission module 112 is operatively coupled to an online billing platform 102, which can be used either by a user or any healthcare professional to submit the pending insurance claims. The user may be either a patient or the parents, caregivers, or guardians of the patient. The healthcare professional may be any person who is allotted the responsibility to submit the insurance claims.

The data collector 108 extracts user details 108 and insurance claims details 110 from memory 106 of the online billing platform 102. User details 108 include essential information about patients or healthcare providers, such as personal identification codes, contact information, and medical history or treatment preferences. The insurance claims details 110 covers the financial and administrative aspects which include the coding for medical procedures, treatment descriptions, dates of session, charges for each session, and any other relevant data that might be needed to submit a claim to an insurance payer.

The plurality of reasons for which one or more payers may reject or deny insurance claims incorporates various issues related to the accuracy and completeness of the submitted information. One common reason is the presence of inappropriate or insufficient (i.e., inaccurate) information within the insurance claim form. This can include errors in patient details, such as incorrect identification numbers or misspelled names, and inaccuracies in the coding of medical procedures or diagnoses. Such discrepancies can prevent the insurance company from accurately processing the claim and matching it with the patient's insurance coverage. Another critical reason for claim denial is insurance expiration. If a claim is submitted after the patient's insurance policy has lapsed, the payer may reject the claim outright. This highlights the importance of verifying that the patient's insurance is active and valid at the time of service.

Additionally, claims may be denied if the requested reimbursement amount exceeds the threshold values established by the insurance policy. Insurance plans often have predefined limits for specific procedures or treatments, and claims exceeding these limits may not be fully covered. In such cases, the payer may only partially pay the claim or deny it altogether, depending on the policy terms. Non-covered medical sessions are another frequent cause of claim rejection. Insurance policies typically specify which treatments and services are covered, and any procedures outside this scope may not be reimbursable. General errors in the submission process also contribute to claim denials.

Finally, incomplete information from users whether healthcare providers or patients can lead to claim denials. This can occur if critical details such as patient demographics, treatment dates, or required documentation are missing or incomplete.

In operation 204, an analyzer 116 analyzes an Electronic Remittance Advice (ERA) reconciliation data 126 provided by the payer. The ERA data 126 helps in identifying discrepancies in expected and actual payments.

The data collected from the data collector 114 along with the ERA data 126 is analyzed by the analyzer 116, integrated within the bulk insurance claim re-submission module 112.

Electronic Remittance Advice (ERA) data 126 is a digital document that provides detailed information about the payment or non-payment of medical insurance claims. The ERA data 126 is a critical component for processing insurance claims and maintaining payment structure between healthcare providers and insurance payers. ERA data 126 includes comprehensive details such as the amount paid for each claim, any adjustments made, and the reasons for these adjustments. This information helps healthcare providers understand the financial outcomes of the insurance claims submitted by them. Additionally, ERA data 126 includes patient and service details like medical session details, and so on, which are used to match the payment information with the corresponding insurance claims, ensuring accurate reconciliation. The ERA data 126 also provides payer identification and explanations for any claim denials or partial payments, allowing providers to address issues and make necessary corrections.

The analyzer 116 employs machine learning algorithms to determine patterns in ERA data 126, enabling it to detect trends and anomalies that might indicate potential issues in future claim submissions. By analyzing historical ERA data 126, the analyzer 116 can learn from historical claims and identify factors that commonly lead to denials or adjustments. This predictive capability helps healthcare providers anticipate problems before they occur, improving the accuracy and efficiency of the claims process and reducing the likelihood of future claim rejections or delays.

In operation 206, a payment checker 120 identifies the payment status of each pending insurance claim and the reason behind non-payment of the insurance claim.

Once the analysis is done using the analyzer 116, the generated insights are transferred to the payment checker 120 which utilizes NLP (Natural Language Processing) techniques using a NLP (Natural Language Processor) 118.

The payment status of identified pending insurance claims incorporates various issues that can affect the full reimbursement of claims submitted to insurance companies. Firstly, claims categorized as “not paid” may be due to several reasons. These include inappropriate or insufficient information (i.e., inaccurate information), such as incorrect patient details or missing documentation, which can prevent the insurance company from processing the claim accurately. Insurance expiration is another critical factor; claims submitted after the patient's insurance policy has lapsed are automatically denied. Additionally, claims might be rejected if the requested reimbursement amount exceeds the threshold values established by the insurance policy, which specify the maximum payable amount for certain services. Non-covered medical sessions also lead to non-payment, where treatments or procedures provided are not included in the patient's insurance plan, thus ineligible for reimbursement. General errors, such as coding mistakes or administrative errors, and incomplete information from users, like missing patient identification or service dates, further contribute to the denial of claims.

Secondly, some unpaid insurance claims are classified as “partially paid.” This can occur when certain elements of the claim are not covered under the insurance policy, such as specific medical sessions or treatments not included in the coverage plan. In these cases, the insurance company pays only for the covered portions, leaving the non-covered parts unpaid. Additionally, partial payment may result when the amount claimed exceeds the policy's threshold values. Insurance policies often set limits on the maximum amounts payable for certain treatments, and any amounts claimed beyond these limits are not reimbursed. In these situations, the insurer pays up to the policy limit, and the excess remains the responsibility of the patient or healthcare provider.

In operation 208, an insurance claim modifier 122 to implement the modifications to ensure that the insurance claims meet the necessary criteria for approval upon bulk re-submission.

After automatically categorizing the pending insurance claims under different categories like non-paid or partially paid, the pending insurance claim forms are passed on to the insurance claim modifier 122 where the pending insurance claim forms are modified either manually or automatically based on the nature of the change that needs to be made. For instance, the pending insurance claim forms may be CMS 1500 form for out-patients, and CMS 1450 form for in-patients, and so on. The insurance claim modifier 122 is operatively coupled to the payment checker 120 and is integrated within the bulk insurance claim re-submission module 112.

In the process of handling insurance claims, errors and discrepancies often result in claims being marked as pending and requiring modifications. These issues are automatically detected and flagged using the analyzed insights of ERA reconciliation data 126. The ERA data 126 provides a detailed explanation of payments, adjustments, and denials to identify specific reasons why a claim may not have been processed successfully. The flagged discrepancies could include incorrect patient details, incomplete service descriptions, or missing documentation, which need to be addressed to ensure proper claim processing.

To resolve these issues, several modifications may be necessary for the pending insurance claims. For instance, adding or updating documentation which may involve submitting additional medical records, updated patient information, or detailed treatment descriptions that were initially omitted or inadequately provided. Such documentation supports the validity of the claim and provides the payer with the necessary information to process the reimbursement.

Further, filling in any missing or incomplete information blocks is essential for accurately processing the insurance claim. This could include ensuring that all required personal details, such as patient names, addresses, and insurance policy numbers, are accurately filled out. Incomplete patient identification or missing insurance details can lead to delays or denials, as the payer may not have enough information to verify the claim's legitimacy.

Lastly, correcting any errors or omissions in the original insurance claim submission is critical. This involves rectifying incorrect patient data, such as wrong birth dates or insurance numbers, and addressing any inaccuracies in the coding of medical procedures or diagnoses. These corrections ensure that the claim accurately reflects the services provided and aligns with the patient's insurance coverage, facilitating approval and payment process.

In operation 210, a bulk uploader 124 automatically re-submits all the modified pending claims to the one or more payers in a single bulk upload, ensuring that all the corrected insurance claims are accurately reflected in the submission

Lastly, the pending insurance claims after being modified by the insurance claim modifier 122 are passed on to the bulk uploader 124, which is operatively coupled to the bulk insurance claim re-submission module 112 and the online billing platform 102. The bulk uploader 124 uploads all the modified pending insurance claims at one go. This helps in reducing the manual task of sending insurance claims one by one manually. Also, the automation of insurance claims analysis and sharing with the payer reduces a lot of manual hard work and chances of error.

The bulk uploader 124 further generates a report summarizing the status of all insurance claims and provides a comprehensive overview of the healthcare provider's financial interactions with insurance payers. This report includes detailed categorizations of claims, such as those pending, rejected, or approved, offering insights into the current state of each claim. For pending claims, the report outlines the specific issues identified, such as missing information or documentation errors, and details the actions taken for reconciliation and re-submission. By highlighting the modifications made, such as correcting data inaccuracies or adding necessary documents, the report serves as a critical tool for tracking the progress of claims resolution and ensuring that all claims are accurately processed for timely reimbursement.

The pending insurance claims along with the errors due to which they have been denied or rejected are made visible to the user on a user interface 104 integrated within the online billing platform 102.

The automated bulk insurance claims re-submission system 100 further comprises a notification module 128, operatively coupled to the online billing platform 102 and the bulk insurance claim re-submission module 112. The notification module 128 is an essential component in managing healthcare insurance claims, as it proactively informs healthcare providers and users about the status of their pending claims. The notification module 128 is designed to deliver timely updates on various stages of the claims process, ensuring that all parties are aware of any actions needed to expedite claim resolution. Notifications can include alerts for claims that require immediate attention, such as those nearing deadlines or those flagged for potential issues like insufficient documentation or incorrect information. Additionally, the notification module 128 can send reminders for submitting additional documentation, thereby reducing the risk of delays or denials due to incomplete submissions. By keeping healthcare providers and users informed, the notification module 128 enhances communication and efficiency, facilitating a smoother claims management process and helping to ensure prompt reimbursement for services rendered.

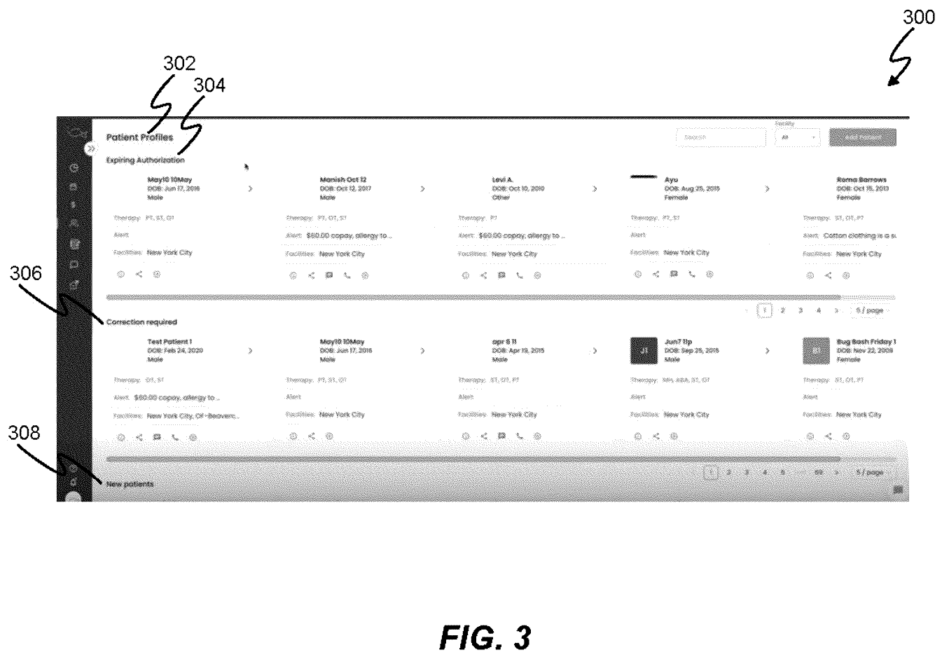

FIG. 3 depicts an exemplary user interface 300 disclosing the user profile categorized based on various parameters.

The user interface 300 shows the user profile, displaying the user details 108 and insurance claim details 110 of the user which are stored in the memory of the online billing platform 102. The patient profile 302 includes a plurality of user profiles each categorized into different categories and placed under the respective categories. These categories include: ‘Expiring Authorization’ 304, ‘Correction Required’ 306, ‘New Patients’308, and so on.

The ‘Expiring Authorization’ 304 profile includes details of all those users whose insurance authorization is about to expire. The expiring authorization refers to a situation where a prior authorization, granted by an insurance company for a specific medical service or procedure, is nearing its expiration date. Prior authorization is a requirement from the insurance company that the healthcare provider obtains approval before providing certain services to ensure that they are covered under the patient's insurance plan. This authorization typically has a validity period, during which the approved services must be reduced.

If the services are not provided within this authorized time frame, or if the authorization expires before the services are completed, the insurance company may deny payment for those services. In such cases, the healthcare provider may need to request a renewal or extension of the authorization to ensure that the services are covered and the claim is not denied due to an expired authorization. Managing expiring authorizations is crucial for healthcare providers to secure reimbursement for the services they provide.

The ‘Correction Required’ 306 includes the details of profiles of all those users whose insurance claims are rejected or denied by the payer. The rejection or denial of the insurance claim may be due to any of the reasons like inappropriate or insufficient information, the amount exceeding the threshold values, non-covered medical sessions, general errors, and incomplete information from users. The ‘New Patients’308 includes the details of all the new users registered to the medical center.

The categories ‘Expiring Authorization’ 304, ‘Correction Required’ 306, and ‘New Patients’ 308 include user details 108 like name, DOB of the user, and therapy center address of the user, and insurance claims details 110 like any substance or medicine allergic to the user, the amount to be paid by the user i.e., copay. The copay (copayment) is a fixed amount that a patient is required to pay out-of-pocket for a specific healthcare service or prescription medication at the time the service is provided. The copay is a form of cost-sharing between the insurance company and the patient, where the patient pays a portion of the cost, and the insurance company covers the rest.

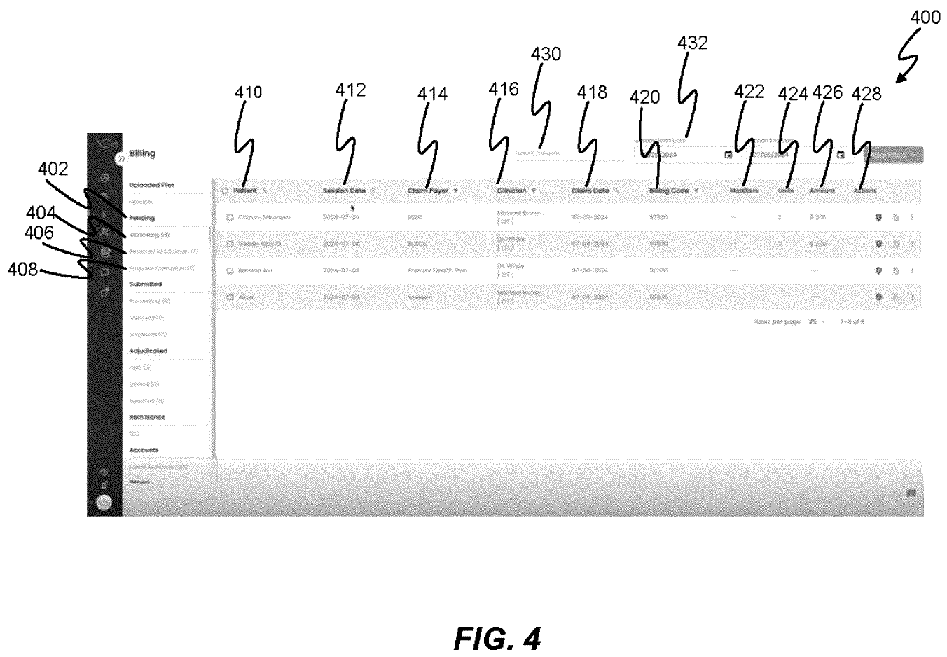

FIG. 4 depicts an exemplary user interface 400 disclosing the list of pending insurance claims.

The user interface 400 discloses the list of all the users whose insurance claims are at the pending stage, i.e., their insurance claims are either rejected or denied by the insurance company i.e., the payer. The user interface 400 includes a tab ‘Pending’ 402 which has three different sub-sections namely, ‘Reviewing’ 404, ‘Returned to Clinician’ 406, and ‘Requires Correction’ 408.

Upon clicking on the tab ‘Reviewing’ 404, the list of all the users whose insurance claims are at the pending stage appears. The list contains details such as user name 410, date of session 412, claim payer 414, clinician 416, claim date 418, billing code 420, modifiers 422, units 424, amount 426, and actions 428.

The user name 410 includes the name of the user, and the date of session 412 includes the date on which the medical session is held. The claim payer 414 includes the details of the payer i.e., the insurance company who is making the payment, the clinician 416 includes details of the therapist or the medical expert who is taking medical sessions and the name of the medical sessions. The claim date 418 includes the date on which the insurance claim is sent to the payer for the first time, and the billing code 420 includes the identification code for that insurance claim. Further, the modifier 422 are alpha-numeric codes that provide extra details about the medical session, the units 424 discloses the duration of a medical session, for instance, 15 minutes is equal to 1 unit. The amount 426 depicts the actual charge for that particular medical session, and the actions 428 discloses alert messages, and various other options like refresh, modify, share, and so on.

The list can be accessed either by directly entering the user name in the tab ‘user name’ 430, or using filters like ‘session start date’ and ‘session end date’ 432. Other filters like therapist name, therapy-based, and so on can be used to access the list.

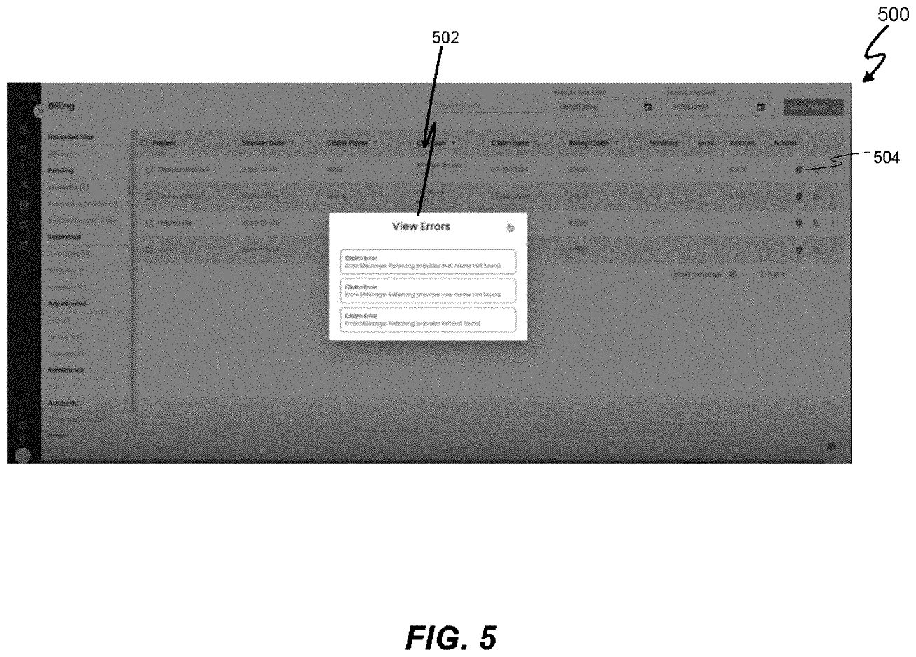

FIG. 5 depicts an exemplary user interface 500 that discloses the reason for the rejection or denial of the pending insurance claim.

The user interface 500 discloses the reason for the rejection or denial of the insurance claim. The reason for the rejection or denial of the insurance claim is automatically detected using the insights provided by the analyzer 116. The analyzer 116 provides insights after analyzing the ERA data 126 and the user detail 108 and insurance claim details 110 stored in the memory 106 of the online billing platform 102.

ERA data 126 provides a detailed record of how submitted insurance claims are processed by payers, including payment adjustments, denials, and the reasons behind them. The analyzer 116 analyzes this data to identify discrepancies between the expected and actual outcomes of claims. For instance, discrepancies may arise from incorrect billing codes, mismatched patient information, missing documentation, or details that do not align with the coverage terms of the insurance policy. The automated analysis highlights these issues, such as claims submitted with outdated patient information or insufficient justification for the medical services provided, which can result in partial payments or denials. By flagging these errors 502 and discrepancies, the healthcare providers and users are notified of the specific areas needing correction, enabling timely and accurate modifications. This process ensures that resubmitted claims are complete and accurate, reducing the likelihood of further delays or denials, and facilitating quicker reimbursements.

The error 502 is shown with a red flag 504. Upon clicking on that red flag 504, the errors on that particular insurance claim are displayed to the healthcare professional on the user interface 500.

The errors 502 generated may be like, for instance, ‘Referring provider first name not found’, ‘Referring provider last name not found, ‘Referring provider NPI not found’, and so on. The healthcare professional looks after these errors 502 and either modifies them manually or automatically using the insurance claim modifier 122, whichever is needed in that situation.

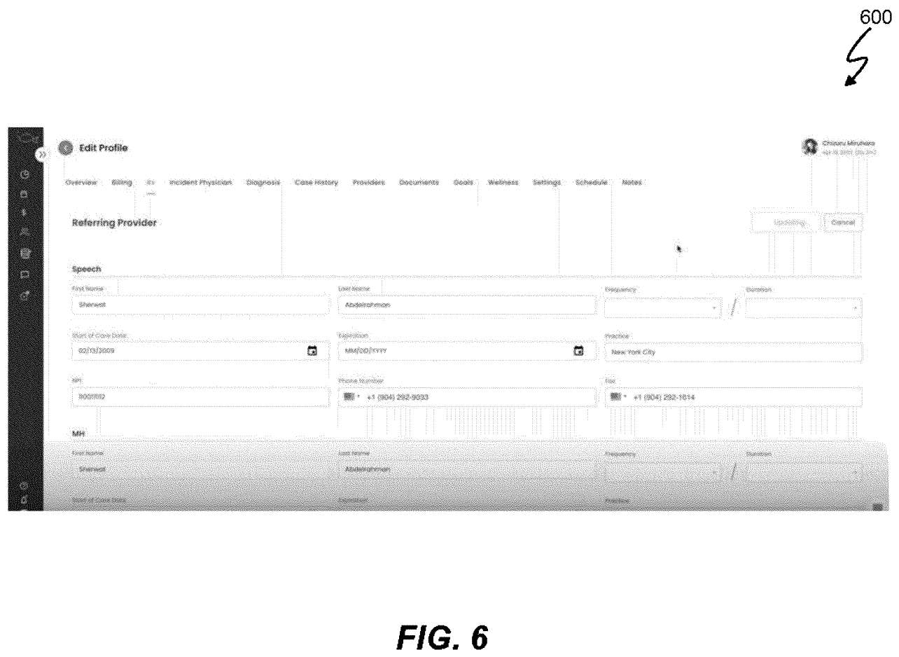

FIG. 6 depicts an exemplary user interface 600 that discloses the modified insurance claim form by the user.

The user interface 600 discloses the modified insurance claim form based on error 502 displayed to the health professional on user interface 500. The changes can be made either manually, or it can be done automatically using the insurance claim modifier 122.

The insurance claim modifier 122 addresses and corrects issues that cause insurance claims to be pending or denied. For instance, the insurance claim modifier 122 may add or update the necessary documentation that supports the pending claim. This may include medical records, detailed patient information, or comprehensive treatment details that provide a clear and complete picture of the services rendered. Accurate and thorough documentation is essential for validating the claim and ensuring that it meets the payer's requirements.

Further, the insurance claim modifier 122 may also be used to fill in any missing or incomplete information blocks crucial for the accurate processing of the claim. This step involves providing or correcting personal details, patient identification, or insurance information that may have been missing or incorrectly entered in the original submission. Accurate data entry is vital to prevent confusion or errors that could lead to claim denials.

Also, the insurance claim modifier 122 may fill in any errors or omissions found in the initial claim submission. This includes correcting inaccuracies in patient data, such as names, dates of birth, or insurance policy numbers, as well as rectifying any coding errors related to the medical procedures or diagnoses. By ensuring all information is accurate and complete, the insurance claim modifier 122 helps in reducing the likelihood of further delays or denials and facilitating quicker and more accurate reimbursement for healthcare providers.

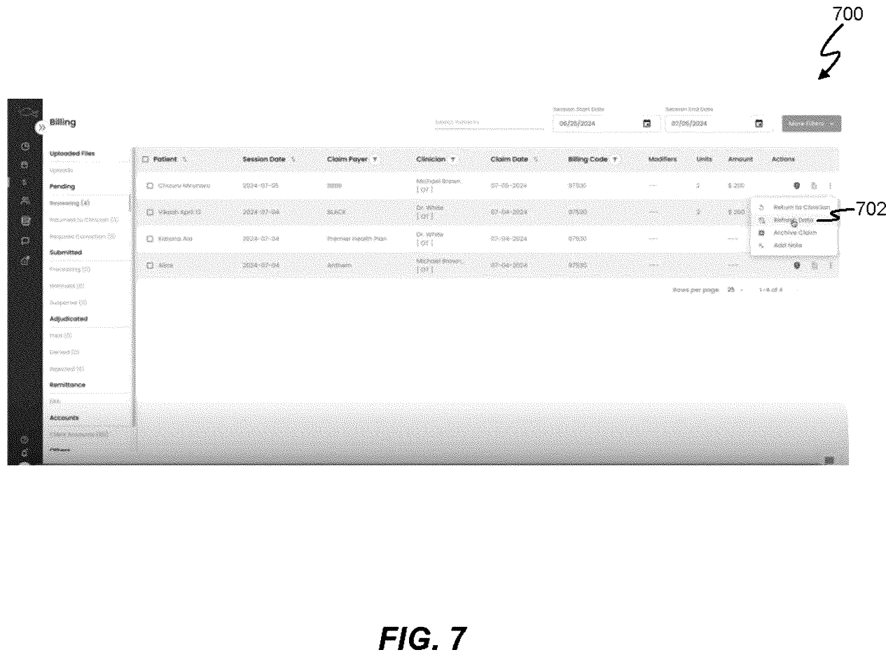

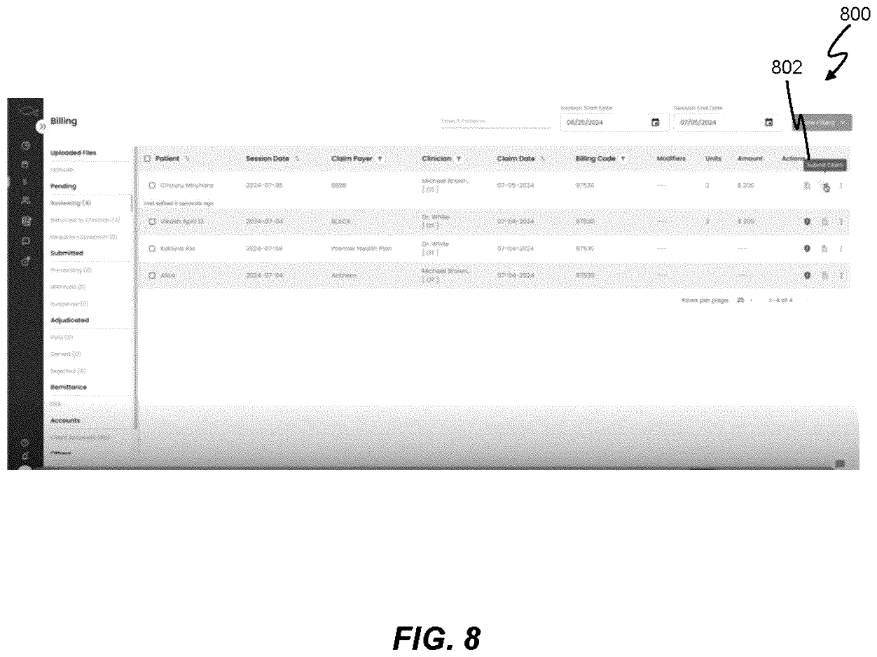

FIGS. 7 and 8 depict exemplary user interfaces that refresh the pending insurance claim form after modification and submit it to the payer respectively.

Upon modification of the insurance claim form using the insurance claim modifier 122, the list of pending insurance claims displayed on the user interface 400 is refreshed by clicking on the tab ‘Refresh’ 702. By clicking on the tab ‘Refresh’ 702, the pending insurance claims get updated and get ready for submission.

Finally, in user interface 800, the user can click on the ‘Submit’ tab to submit the modified insurance claim form to get the reimbursement done.

Thus, because, for example, medical service providers are often reluctant to transition from one EMR system to another. The systems discussed herein provide a technical solution that allows medical service providers to overcome technical barriers and transition from one EMR system to another.

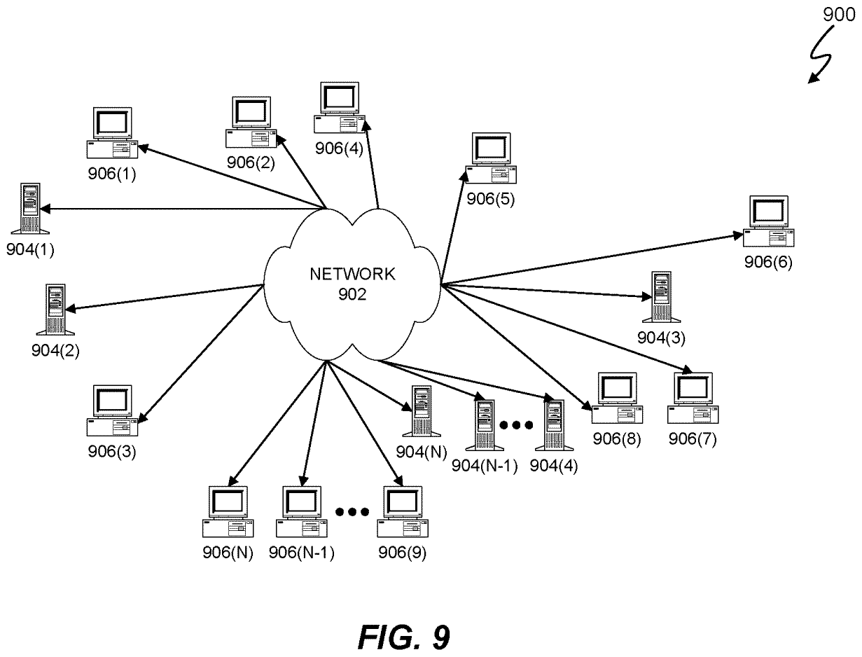

FIG. 9 is a block diagram illustrating a network environment in which a bulk insurance claims re-submission system 100 and process 200 may be practiced. Network 902 (e.g. a private wide area network (WAN) or the Internet) includes several networked server computer systems 904(1)-(N) that are accessible by client computer systems 906(1)-(N), where N is the number of server computer systems connected to the network. Communication between client computer systems 906(1)-(N) and server computer systems 904(1)-(N) typically occurs over a network, such as a public switched telephone network over asynchronous digital subscriber line (ADSL) telephone lines or high-bandwidth trunks, for example, communications channels providing T1 or OC3 service. Client computer systems 906(1)-(N) typically access server computer systems 904(1)-(N) through a service provider, such as an internet service provider (“ISP”) by executing application-specific software, commonly referred to as a browser, on one of client computer systems 906(1)-(N).

Client computer systems 906(1)-(N) and server computer systems 904(1)-(N) are specialized computers programmed to improve conventional computer systems to implement and utilize the bulk insurance claims re-submission system 100 and process 200. The type of computer system that can be specially programmed to implement and utilize the user guidance system 100 and process 200 using the real-time tutor bulk insurance claims re-submission system 100 and process 200 includes a mainframe, a mini-computer, a personal computer system including notebook computers, a wireless, mobile computing device (including personal digital assistants, smartphones, and tablet computers). These computer systems are typically designed to provide computing power to one or more users locally or remotely. Each computer system may also include one or a plurality of input/output (“I/O”) devices coupled to the system processor to perform specialized functions. Tangible, non-transitory memories (also referred to as “storage devices”) such as hard disks, compact disk (“CD”) drives, digital versatile disk (“DVD”) drives, and magneto-optical drives may also be provided, either as an integrated or peripheral device. In at least one embodiment, the bulk insurance claims re-submission system 100 and process 200 can be implemented using code stored in a tangible, non-transient computer-readable medium and executed by one or more processors. In at least one embodiment, the bulk insurance claims re-submission system 100 and process 200 can be implemented completely in hardware using, for example, logic circuits and other circuits including field programmable gate arrays.

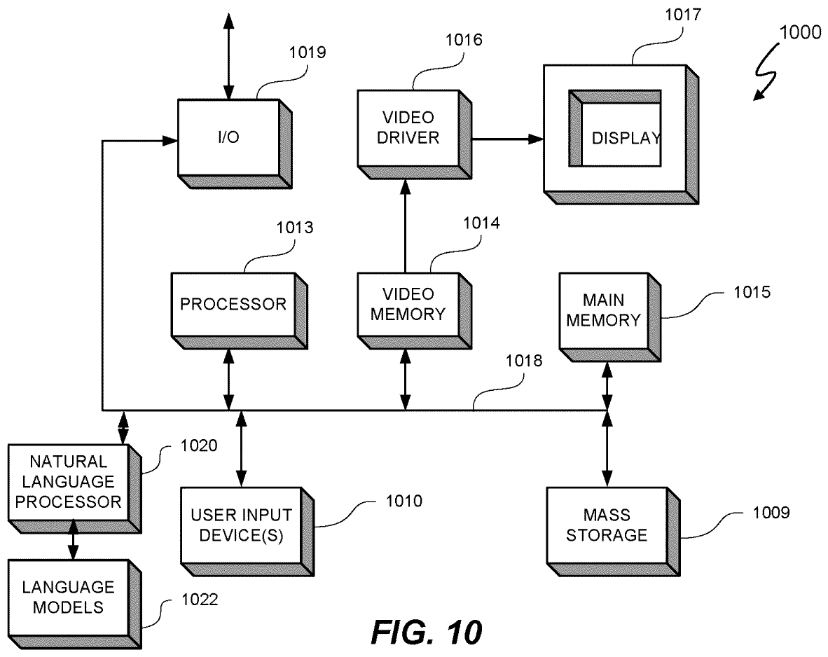

Embodiments of the bulk insurance claims re-submission system 100 and process 200 can be implemented on a computer system such as a special-purpose, special-programmed computer 1000 illustrated in FIG. 10. Input user device(s) 1010, such as a keyboard and/or mouse, are coupled to a bi-directional system bus 1018. The input user device(s) 1010 are for introducing user input to the computer system and communicating that user input to processor 1013. The computer system of FIG. 10 generally also includes a non-transitory video memory Y14, non-transitory main memory 1015, and non-transitory mass storage 1009, all coupled to bi-directional system bus 1018 along with input user device(s) 1010 and processor 1013. The mass storage 1009 may include fixed and removable media, such as a hard drive, one or more CDs or DVDs, solid state memory including flash memory, and other available mass storage technology. Bus 1018 may contain, for example, 32 of 64 address lines for addressing video memory 1014 or main memory 1015. The system bus 1018 also includes, for example, an n-bit data bus for transferring DATA between and among the components, such as CPU 1009, main memory 1015, video memory 1014, and mass storage 1009, where “n” is, for example, 32 or 64. Alternatively, multiplex data/address lines may be used instead of separate data and address lines.

I/O device(s) 1019 may provide connections to peripheral devices, such as a printer, and may also provide a direct connection to a remote server computer system via a telephone link or to the Internet via an ISP. I/O device(s) 1019 may also include a network interface device to provide a direct connection to a remote server computer system via a direct network link to the Internet via a POP (point of presence). Such connection may be made using, for example, wireless techniques, including digital cellular telephone connection, Cellular Digital Packet Data (CDPD) connection, digital satellite data connection, or the like. Examples of I/O devices include modems, sound and video devices, and specialized communication devices such as the aforementioned network interface.

Computer programs and data are generally stored as code in a non-transient computer-readable medium such as flash memory, optical memory, magnetic memory, compact disks, digital versatile disks, and any other type of memory. The computer program is loaded from a memory, such as mass storage 1009, into main memory 1015 for execution. Computer programs may also be in the form of electronic signals modulated in accordance with the computer program and data communication technology when transferred via a network. In at least one embodiment, Java applets or any other technology is used with web pages to allow a user of a web browser to make and submit selections and allow a client computer system to capture the user selection and submit the selection data to a server computer system.

The processor 1013, in one embodiment, is a microprocessor manufactured by Motorola Inc. of Illinois, Intel Corporation of California, or Advanced Micro Devices of California. However, any other suitable single or multiple microprocessors or microcomputers may be utilized. Main memory 1015 consists of dynamic random access memory (DRAM). Video memory 1014 is a dual-ported video random access memory. One port of the video memory 1014 is coupled to the video amplifier 1016. The video amplifier 1016 is used to drive the display 1017. Video amplifier 1016 is well-known in the art and may be implemented by any suitable means. This circuitry converts pixel DATA stored in video memory 1014 to a raster signal suitable for use by display 1017. Display 1017 is a type of monitor suitable for displaying graphic images.

The computer system described above is for purposes of example only. The bulk insurance claims re-submission system 100 and process 200 may be implemented in any type of computer system programming or processing environment. It is contemplated that the bulk insurance claims re-submission system 100 and process 200 might be run on a stand-alone computer system, such as the one described above. The bulk insurance claims re-submission system 100 and process 200 might also be run from a server computer systems system that can be accessed by a plurality of client computer systems interconnected over an intranet network. Finally, the bulk insurance claims re-submission system 100 and process 200 may be run from a server computer system that is accessible to clients over the Internet.

Although embodiments have been described in detail, it should be understood that various changes, substitutions, and alterations can be made herein without departing from the spirit and scope of the invention as defined by the appended claims.

What is claimed is:

1. A method of transitioning from one technical electronic medical record (EMR) system to another EMR system using automatic bulk re-submission of pending insurance claims, the method comprises:
executing code by a computer system to enable transition from one EMR system to another EMR system by causing the computer system to perform operations comprising:
uploading insurance medical records and insurance claims from a first electronic medical records system to the computer system to facilitate transitioning from the first electronic medical records system to a second electronic medical records system;
extracting past insurance claims that were previously submitted and not fully paid by a payer due to at least one of a plurality of reasons including denial and rejection, wherein the previously submitted and unpaid insurance claims are classified as pending insurance claims;
analyzing an Electronic Remittance Advice (ERA) reconciliation data provided by the payer for pending insurance claims, wherein the ERA data helps in identifying discrepancies in the pending insurance claims;
identifying the payment status of each pending insurance claim and the reason behind non-payment of that pending insurance claim;
implementing a set of modifications to each of the pending insurance claims to meet the necessary criteria for approval of the pending insurance claims upon bulk re-submission;
automatically re-submitting the modified pending insurance claims to the payer in a single bulk upload, ensuring that all the corrected insurance claims are accurately reflected in the re-submitted insurance claims.

2. The method of claim 1 wherein the at least one of a plurality of reasons due to which the payer reject the insurance claims includes inaccurate information, insurance expiration, the amount exceeding the threshold values, non-covered medical sessions, general errors, and incomplete information from users.

3. The method of claim 1 wherein the ERAs provide detailed information about the payment, such as the amount paid, any adjustments made, and the reason codes.

4. The method of claim 1 wherein the payment status of the identified pending insurance claims further includes:
not paid due to inaccurate information, insurance expiration, the amount exceeding the threshold values, non-covered medical sessions, general errors, and incomplete information from users; and
partially paid insurance claims due to non-covered medical sessions, and the amount exceeding the threshold values.

5. The method of claim 1 wherein the errors and discrepancies due to which the insurance claim is pending and requires modifications are automatically detected and flagged based on the analyzed ERA reconciliation data.

6. The method of claim 1 wherein the modifications needed for fixing the pending insurance claims include:
updating documentation that supports the pending insurance claim, such as medical records, patient information, and treatment details;
filling in any incomplete information blocks that are required for accurate insurance claim processing, such as personal details, patient identification, and insurance details; and
correcting any errors and omissions found in the original insurance claim submission, such as incorrect patient data.

7. The method of claim 1 further comprises:
generating a report summarizing the status of all insurance claims, including pending claims, as well as the actions taken for reconciliation and re-submission.

8. The method of claim 1 wherein the payer is an insurance company, provided with a unique ID code.

9. The method of claim 1 further comprising:
executing the code by the one or more processors to cause the computer system to perform further operations including notifying healthcare providers and users about the status of pending insurance claims, including notifications for insurance claims requiring immediate attention or additional documentation.

10. A system for transitioning from one technical electronic medical record (EMR) system to another EMR system using automatic bulk re-submission of pending insurance claims, the system comprising:
one or more processors of a computer system; and
a memory, coupled to the one or more processors, that stores code and execution of the code by the one or more processors causes the computer system to perform operations comprising:
uploading insurance medical records and insurance claims from a first electronic medical records system to the computer system to facilitate transitioning from the first electronic medical records system to a second electronic medical records system;
extracting past insurance claims that were previously submitted and not fully paid by a payer due to at least one of a plurality of reasons including denial and rejection, wherein the previously submitted and unpaid insurance claims are classified as pending insurance claims;
analyzing an Electronic Remittance Advice (ERA) reconciliation data provided by the payer for pending insurance claims, wherein the ERA data helps in identifying discrepancies in the pending insurance claims;
identifying the payment status of each pending insurance claim and the reason behind non-payment of that pending insurance claim;
implementing a set of modifications to each of the pending insurance claims to meet the necessary criteria for approval of the pending insurance claims upon bulk re-submission; and
automatically re-submitting the modified pending insurance claims to the payer in a single bulk upload, ensuring that all the corrected insurance claims are accurately reflected in the re-submitted insurance claims.

11. The system of claim 10 wherein the pending insurance claims are visible to the user or healthcare provider on a user interface integrated within the online billing platform.

12. The system of claim 10 wherein the one or more databases store historical claim data, patient records, and documentation necessary for verifying and supporting insurance claims.

13. The system of claim 10 wherein the analyzer utilizes machine learning algorithms to identify patterns in ERA reconciliation data, helping to predict potential issues in future claim submissions.

14. The system of claim 10 wherein execution of the code by the one or more processors causes the computer system to perform further operations comprising:
notifying healthcare providers and users about the status of pending insurance claims, including notifications for insurance claims requiring immediate attention or additional documentation.

15. The system of claim 10 wherein the at least one of a plurality of reasons due to which the payer reject the insurance claims includes inaccurate information, insurance expiration, the amount exceeding the threshold values, non-covered medical sessions, general errors, and incomplete information from users.

16. The system of claim 10 wherein the ERAs provide detailed information about the payment, such as the amount paid, any adjustments made, and the reason codes.

17. The system of claim 10 wherein the payment status of the identified pending insurance claims further includes:
not paid due to inaccurate information, insurance expiration, the amount exceeding the threshold values, non-covered medical sessions, general errors, and incomplete information from users; and
partially paid insurance claims due to non-covered medical sessions, and the amount exceeding the threshold values.

18. The system of claim 10 wherein the errors and discrepancies due to which the insurance claim is pending and requires modifications are automatically detected and flagged based on the analyzed ERA reconciliation data.

19. The system of claim 10 wherein the modifications needed for fixing the pending insurance claims include:
updating documentation that supports the pending insurance claim, such as medical records, patient information, and treatment details;
filling in any incomplete information blocks that are required for accurate insurance claim processing, such as personal details, patient identification, and insurance details; and
correcting any errors and omissions found in the original insurance claim submission, such as incorrect patient data.

20. The system of claim 10 further comprises:
generating a report summarizing the status of all insurance claims, including pending claims, as well as the actions taken for reconciliation and re-submission.

21. The system of claim 10 wherein the payer is an insurance company, provided with a unique ID code.
